(19) United States
(12) Patent Application Publication
(10) Pub. No.: US 2025/0319457 A1
(43) Pub. Date: Oct. 16, 2025

(54) CATALYST FOR THE MANUFACTURE OF ACRYLONITRILE

(71) Applicant: INEOS Europe AG, Rolle (CH)

(72) Inventors: Michael J. Seely, Plainfield, IL (US); Sean S. Lin, Naperville, IL (US); Leighta M. Johnson, Batavia, IL (US); Robert A. Gustaferro, Naperville, IL (US)

(73) Assignee: INEOS Europe AG, Rolle (CH)

(21) Appl. No.: 18/866,139

(22) PCT Filed: May 19, 2023
(86) PCT No.: PCT/IB2023/055203

§ 371 Date: Nov. 15, 2024

Related U.S. Application Data
(60) Provisional application No. 63/344,482, filed on May 20, 2022.
(60) Provisional application No. 63/476,635, filed on Dec. 21, 2022.

(51) Int. Cl. B01J 23/887; B01J 23/00; C07C 253/26

(52) U.S. Cl. CPC B01J 23/8878; B01J 23/002; C07C 253/26; B01J 2523/14; B01J 2523/22; B01J 2523/3712; B01J 2523/54; B01J 2523/67; B01J 2523/68; B01J 2523/842; B01J 2523/847

(57) ABSTRACT

The invention relates to catalyst compositions comprising a complex of catalytic oxides comprising molybdenum, bismuth, cerium, iron, chromium, at least one element of group A, at least one element of group B, and optionally at least one element of group C wherein the relative ratios of these elements are represented by Formula (1): Mo₁₂BiₐCe_{b}Fe_{c}Cr_{d}AₑB_{f}C_{g}Oₓ. The invention also relates to a process for the ammoxidation of an olefin comprising reacting in the vapor phase at an elevated temperature and pressure the olefin with a molecular oxygen containing gas and ammonia in the presence of the catalyst composition.

### FIELD OF THE INVENTION

The present invention relates to an improved catalyst composition useful for the ammoxidation of an unsaturated hydrocarbon to the corresponding unsaturated nitrile. In certain embodiments, the present invention is directed to an improved process and catalyst for the ammoxidation of propylene to acrylonitrile and/or isobutylene to methacrylonitrile. More specifically, the invention relates to an improved ammoxidation catalyst comprising a complex of catalytic oxides comprising molybdenum, bismuth, cerium, iron, chromium, at least one element of group A, at least one element of group B, and optionally at least one element of group C; wherein group A consists of sodium, potassium, rubidium, and cesium; wherein group B consists of nickel, cobalt, manganese, zinc, magnesium, calcium, strontium, cadmium, and barium; and wherein group C consists of silver, gold, ruthenium, rhodium, palladium, osmium, iridium, platinum, and mercury.

The present invention further relates to an improved process and catalyst for the ammoxidation of propylene to acrylonitrile and/or isobutylene to methacrylonitrile utilizing a selective catalyst useful for the production of the unsaturated nitrile in high yield. For example, catalysts of the present invention exhibit a combination of higher overall conversion of propylene and high selectivity to acrylonitrile compared to similar catalysts.

### BACKGROUND OF THE INVENTION

Catalysts containing oxides of iron, bismuth and molybdenum, promoted with suitable elements, for use in the conversion (i.e., ammoxidation) of propylene at elevated temperatures in the presence of ammonia and a source of molecular oxygen (e.g., air) to manufacture acrylonitrile are known.

A need exists for continued improvement in catalysts useful for the conversion of an olefin to the corresponding unsaturated nitrile (e.g., amoxidation of propylene to manufacture acrylonitrile).

An object of the instant invention is a catalyst composition comprising a complex of catalytic oxides comprising a unique combination of relative ratios of the elements, offering better performance in the catalytic ammoxidation of an unsaturated hydrocarbon to the corresponding unsaturated nitrile. For example, propylene, isobutylene or mixtures thereof, to acrylonitrile, methacrylonitrile and mixtures thereof, respectively.

Another object of the instant invention is to provide a process for the ammoxidation of an olefin comprising reacting in the vapor phase at an elevated temperature and pressure the olefin with a molecular oxygen containing gas and ammonia in the presence of a catalyst comprising a complex of catalytic oxides.

The present invention is also directed to processes for the conversion of an olefin selected from the group consisting of propylene, isobutylene or mixtures thereof, to acrylonitrile, methacrylonitrile and mixtures thereof, respectively, by reacting in the vapor phase at an elevated temperature and pressure the olefin with a molecular oxygen containing gas and ammonia in the presence of a catalyst comprising a complex of catalytic oxides as described herein.

### BRIEF SUMMARY OF THE INVENTION

The present invention is directed to an improved catalyst and process for the ammoxidation of an unsaturated hydrocarbon to the corresponding unsaturated nitrile. For example, propylene and/or isobutylene to acrylonitrile and/or methacrylonitrile, respectively.

In one embodiment, a catalyst composition comprises a complex of catalytic oxides comprising molybdenum, bismuth, cerium, iron, chromium, at least one element of group A, at least one element of group B, and optionally at least one element of group C, wherein group A consists of sodium, potassium, rubidium, and cesium; wherein group B consists of nickel, cobalt, manganese, zinc, magnesium, calcium, strontium, cadmium, and barium; wherein group C consists of silver, gold, ruthenium, rhodium, palladium, osmium, iridium, platinum, and mercury; and wherein the relative ratios of these elements are represented by Formula (1):

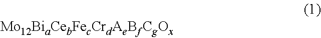

wherein:
   a is from about 0.05 to about 4;
   b is from about 0.01 to about 3;
   c is from about 0.01 to about 4;
   d is from about 0.01 to about 2;
   e is from about 0.01 to about 2;
   f is from about 0.01 to about 10;
   g is from about 0 to about 0.2;
   x is a number determined by the valence requirement of the other elements present; and
wherein c/d is from about 7 to about 12.

In another embodiment, a catalyst composition comprises a complex of catalytic oxides comprising molybdenum, bismuth, cerium, iron, chromium, at least one element of group A, at least one element of group B, and optionally at least one element of group C, wherein group A consists of sodium, potassium, rubidium, and cesium; wherein group B consists of nickel, cobalt, manganese, zinc, magnesium, calcium, strontium, cadmium, and barium; wherein group C consists of silver, gold, ruthenium, rhodium, palladium, osmium, iridium, platinum, and wherein the relative ratios of these elements are represented by Formula (1):

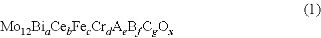

wherein:
   a is from about 0.05 to about 4;
   b is from about 0.3 to about 3;
   c is from about 0.01 to about 4;
   d is from about 0.01 to about 2;
   e is from about 0.01 to about 2;
   f is from about 0.01 to about 10;
   g is from about 0 to about 0.2;
   x is a number determined by the valence requirement of the other elements present; and
wherein c/d is from about 2 to about 17.

In some embodiments, the catalyst composition exhibits a scheelite (m-phase+t-phase):β-MMoO₄ ratio of no greater than about 0.3, wherein amounts of m-phase, t-phase and β-MMoO₄ phase are determined using X-ray diffraction and a modified Rietveld analysis model.

For example, in one embodiment, a catalyst composition comprises a complex of catalytic oxides comprising molybdenum, bismuth, cerium, iron, chromium, at least one element of group A, at least one element of group B, and optionally at least one element of group C, wherein group A consists of sodium, potassium, rubidium, and cesium; wherein group B consists of nickel, cobalt, manganese, zinc, magnesium, calcium, strontium, cadmium, and barium; wherein group C consists of silver, gold, ruthenium, rhodium, palladium, osmium, iridium, platinum, and wherein the relative ratios of these elements are represented by Formula (1):

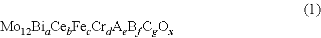

wherein:
   a is from about 0.05 to about 4;
   b is from about 0.01 to about 3;
   c is from about 0.01 to about 4;
   d is from about 0.01 to about 2;
   e is from about 0.01 to about 2;
   f is from about 0.01 to about 10;
   g is from about 0 to about 0.2;
   x is a number determined by the valence requirement of the other elements present; and
wherein c/d is from about 2 to 36 and the catalyst composition exhibits a scheelite (m-phase+t-phase):β-MMoO₄ ratio of no greater than about 0.3, wherein amounts of m-phase, t-phase and β-MMoO₄ phase are determined using X-ray diffraction and a modified Rietveld analysis model.

Another embodiment is directed to a process for the ammoxidation of an olefin. The process comprises reacting in the vapor phase at an elevated temperature and pressure the olefin with a molecular oxygen containing gas and ammonia in the presence of the catalyst comprising a complex of catalytic oxides comprising molybdenum, bismuth, cerium, iron, chromium, at least one element of group A, at least one element of group B, and optionally at least one element of group C.

Other objects and features will be in part apparent and in part pointed out hereinafter.

### DETAILED DESCRIPTION OF THE INVENTION

The instant invention is directed to a catalyst composition comprising a complex of catalytic oxides comprising a unique combination of relative ratios of the elements, offering better performance in the catalytic ammoxidation of an unsaturated hydrocarbon to the corresponding unsaturated nitrile. For example, propylene, isobutylene or mixtures thereof, to acrylonitrile, methacrylonitrile and mixtures thereof, respectively.

The catalyst composition described herein (i.e., apart from any optional support) is a complex of catalytic oxides of molybdenum, bismuth, cerium, iron, chromium, at least one element of group A, at least one element of group B, and optionally at least one element of group C. Group A consists of sodium, potassium, rubidium, and cesium. Group B consists of nickel, cobalt, manganese, zinc, magnesium, calcium, strontium, cadmium, and barium. Group C consists of silver, gold, ruthenium, rhodium, palladium, osmium, iridium, platinum, and mercury. In certain embodiments, the iron is in excess with respect to chromium and the excess is controlled within certain ranges as described herein.

In one embodiment, the relative ratios of these elements in the catalyst composition are represented by Formula (1):

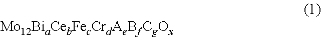

wherein:
   a is from about 0.05 to about 4;
   b is from about 0.01 to about 3;
   c is from about 0.01 to about 4;
   d is from about 0.01 to about 2;
   e is from about 0.01 to about 2;
   f is from about 0.01 to about 10;
   g is from about 0 to about 0.2;
   x is a number determined by the valence requirement of the other elements present; and
wherein c/d is from about 2 to about 36.

In certain embodiments, c/d is from about 2 to about 32, from about 2 to about 28, from about 2 to about 24, from about 2 to about 20, from about 4 to about 36, from about 4 to about 32, from about 4 to about 28, from about 4 to about 24, from about 4 to about 20, from about 7 to about 36, from about 7 to about 32, from about 7 to about 28, from about 7 to about 24, from about 7 to about 20, from about 7 to about 12, from about 7 to about 11.5, from about 7 to about 11, from about 7 to about 10.5, from about 7.5 to about 10.5, from about 8 to about 10.5, or from about 8 to about 10.

In another embodiment, the catalyst composition has a higher cerium content and the relative ratios of these elements in the catalyst composition are represented by Formula (1):

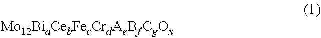

wherein:
   a is from about 0.05 to about 4;
   b is from about 0.3 to about 3;
   c is from about 0.01 to about 4;
   d is from about 0.01 to about 2;
   e is from about 0.01 to about 2;
   f is from about 0.01 to about 10;
   g is from about 0 to about 0.2;
   x is a number determined by the valence requirement of the other elements present; and
wherein c/d is from about 2 to about 17.

In certain embodiments, c/d is from about 2 to about 16, from about 2 to about 14, from about 2 to about 12, from about 2 to about 10, from about 2 to about 8, or from about 2 to about 6.

In these and other embodiments, a (bismuth) is from about 0.05 to about 3.5, from about 0.05 to about 3, from about 0.05 to about 2.5, from about 0.05 to about 2, from about 0.05 to about 1.5, from about 0.05 to about 1, from about 0.1 to about 1, from about 0.15 to about 1, from about 0.2 to about 1, from about 0.25 to about 1, from about 0.3 to about 1, from about 0.35 to about 1, from about 0.4 to about 1, from about 0.05 to about 1.25, from about 0.05 to about 0.75, from about 0.05 to about 0.5, from about 0.05 to about 0.4, from about 0.05 to about 0.3, from about 0.05 to about 0.2, or from about 0.05 to about 0.1.

In these and other embodiments, b (cerium) is from about 0.01 to about 2.5, from about 0.01 to about 2, from about 0.01 to about 1.5, from about 0.02 to about 1.5, from about 0.04 to about 1.5, from about 0.06 to about 1.5, from about 0.08 to about 1.5, from about 0.1 to about 1.5, from about 0.2 to about 1.5, from about 0.3 to about 1.5, from about 0.4 to about 1.5, or from about 0.5 to about 1.5. In certain higher cerium content embodiments, b is from about 0.1 to about 3, from about 0.2 to about 3, from about 0.3 to about 3, from about 0.4 to about 3, from about 0.5 to about 3, from about 0.75 to about 3, from about 1 to about 3, from about 1.5 to about 3, from about 2 to about 3, from about 2.5 to about 3, from about 0.75 to about 2.5, or from about 1 to about 2.5.

In these and other embodiments, c (iron) is from about 0.05 to about 4, from about 0.1 to about 4, from about 0.15 to about 4, from about 0.5 to about 4, from about 0.25 to about 4, from about 0.3 to about 4, from about 0.35 to about 4, from about 0.4 to about 4, from about 0.45 to about 4, from about 0.5 to about 4, from about 0.75 to about 4, from about 1 to about 4, from about 1 to about 3.5, from about 1 to about 3, from about 0.01 to about 3.5, from about 0.01 to about 3, from about 0.01 to about 21, from about 0.01 to about 1, from about 0.01 to about 0.5, from about 0.01 to about 0.4, from about 0.01 to about 0.3, from about 0.01 to about 0.2, from about 0.01 to about 0.1, or from about 0.01 to about 0.05.

In these and other embodiments, d (chromium) is from about 0.01 to about 1.5, from about 0.01 to about 1, from about 0.02 to about 1, from about 0.04 to about 1, from about 0.06 to about 1, from about 0.08 to about 1, from about 0.1 to about 1, from about 0.15 to about 1, from about 0.15 to about 0.5, from about 0.02 to about 2, from about 0.05 to about 2, from about 0.1 to about 2, from about 0.5 to about 2, from about 1 to about 2, or from about 1.5 to about 2.

In these and other embodiments, e (Group A) is from about 0.01 to about 1.5, from about 0.01 to about 1, from about 0.01 to about 0.5, from about 0.01 to about 0.4, from about 0.01 to about 0.3, from about 0.01 to about 0.3, from about 0.02 to about 0.3, from about 0.04 to about 0.3, from about 0.06 to about 0.3, from about 0.08 to about 0.3, from about 0.01 to about 0.3, from about 0.02 to about 2, from about 0.05 to about 2, from about 0.1 to about 2, from about 0.5 to about 2, from about 1 to about 2, or from about 1.5 to about 2.

In these and other embodiments, f (Group B) is from about 0.02 to about 10, from about 0.04 to about 10, from about 0.06 to about 10, from about 0.08 to about 10, from about 0.1 to about 10, from about 0.2 to about 10, from about 0.4 to about 10, from about 0.6 to about 10, from about 0.8 to about 10, from about 1 to about 10, from about 1.5 to about 10, from about 2 to about 10, from about 2.5 to about 10, from about 3 to about 10, from about 3.5 to about 10, from about 4 to about 10, from about 4.5 to about 10, from about 5 to about 10, from about 0.01 to about 8, from about 0.01 to about 7, from about 0.01 to about 6, from about 0.01 to about 5, from about 0.01 to about 4, from about 0.01 to about 3, from about 0.01 to about 2, from about 0.01 to about 1, from about 0.01 to about 0.5, from about 0.01 to about 0.25, from about 0.01 to about 0.2, from about 0.01 to about 0.1, or from about 0.01 to about 0.05.

In some embodiments, if present, g (Group C) is from about 0.01 to about 0.2, from about 0.02 to about 0.2, from about 0.04 to about 0.2, from about 0.06 to about 0.2, from about 0.08 to about 0.2, or from about 0.1 to about 0.2. In an alternative embodiment, g is from about 0 to about 0.15, from about 0 to about 0.1, from about 0 to about 0.05, from about 0 to about 0.04, from about 0 to about 0.03, from about 0 to about 0.02, or from about 0 to about 0.01.

In certain embodiments, A is selected from the group consisting of potassium, rubidium, and cesium. In other embodiments, A is selected from the group consisting of sodium, rubidium, and cesium. In a further embodiment, A is selected from the group consisting of sodium, potassium, and cesium. In a still further embodiment, A is selected from the group consisting of sodium, potassium, and rubidium. In another embodiment, A is selected from the group consisting of potassium, rubidium, and cesium. In one specific embodiment, A is selected from the group consisting of rubidium and cesium.

In certain embodiments, B is selected from the group consisting of nickel, manganese, zinc, magnesium, calcium, strontium, cadmium, and barium. In another embodiment, B is selected from the group consisting of nickel, cobalt, zinc, magnesium, calcium, strontium, cadmium, and barium. In a further embodiment, B is selected from the group consisting of nickel, cobalt, manganese, magnesium, calcium, strontium, cadmium, and barium. In one embodiment, B is selected from the group consisting of nickel, cobalt, manganese, zinc, calcium, strontium, cadmium, and barium. In another embodiment, B is selected from the group consisting of nickel, cobalt, manganese, zinc, magnesium, calcium, cadmium, and barium. In a further embodiment, B is selected from the group consisting of nickel, cobalt, manganese, zinc, magnesium, calcium, strontium, and barium. In still another embodiment, B is selected from the group consisting of nickel, cobalt, manganese, zinc, magnesium, calcium, strontium, and cadmium.

In certain embodiments, if present, C is selected from the group consisting of gold, ruthenium, rhodium, palladium, osmium, iridium, platinum, and mercury. In another embodiment, C is selected from the group consisting of silver, ruthenium, rhodium, palladium, osmium, iridium, platinum, and mercury. In a further embodiment, C is selected from the group consisting of silver, gold, rhodium, palladium, osmium, iridium, platinum, and mercury. In one embodiment, C is selected from the group consisting of silver, gold, ruthenium, palladium, osmium, iridium, platinum, and mercury. In various embodiment, C is selected from the group consisting of silver, gold, ruthenium, rhodium, osmium, iridium, platinum, and mercury. In certain embodiment, C is selected from the group consisting of silver, gold, ruthenium, rhodium, palladium, iridium, platinum, and mercury. In another embodiment, C is selected from the group consisting of silver, gold, ruthenium, rhodium, palladium, osmium, platinum, and mercury. In a further embodiment, C is selected from the group consisting of silver, gold, ruthenium, rhodium, palladium, osmium, iridium, and mercury. In another embodiment, C is selected from the group consisting of silver, gold, ruthenium, rhodium, palladium, osmium, iridium, and platinum

In accordance with some embodiments, one or more specific elements may be excluded from the catalyst composition (i.e., ingredients including the element(s) are not added during preparation of the catalytic oxides component of the catalyst composition). For example, in one embodiment, the catalyst composition does not include potassium. In another embodiment, the catalyst composition does not include rubidium. In a further embodiment, the catalyst composition does not include sodium. In another embodiment, the catalyst composition does not include magnesium. In a still further embodiment, the catalyst composition does not include calcium. Other elements or promoters may be included in the catalyst composition. For example, in some embodiments, the catalyst composition includes one or more of elements selected from the group consisting of phosphorus, tin, indium, antimony, tellurium, lithium, thallium, boron, germanium, and rare earth elements (defined herein as any one of La, Pr, Nd, Pm, Sm, Eu, Gd, Tb, Dy, Ho, Er, Tm, or Yb). For example, in one embodiment, the catalyst composition contains a small amount of phosphorus, which has a beneficial effect on the attrition resistance of the catalyst.

Bismuth, cerium, iron and chromium may be introduced into the catalyst composition in the form of any compound containing the element, such as an oxide or as a salt, which upon calcination will yield the oxides. In certain embodiments, water-soluble salts that are easily dispersed within the catalyst, but form stable oxides upon heat-treating may be used. For example, in one embodiment, sources for introducing these elements include bismuth nitrate, cerium nitrate, ferric nitrate and chromium nitrate.

The molybdenum component of the catalyst composition may be introduced from any molybdenum oxide. However, it is preferred that a hydrolizable or decomposable molybdenum salt be utilized as the source of the molybdenum (e.g., ammonium heptamolybdate).

Other required or optional components (i.e., elements of group A, group B, and group C) and optional elements or promoters of the catalyst composition (e.g., P, Sn, Te, B, Ge, In, or mixtures thereof) may be derived from any suitable source. For example, cobalt, nickel and magnesium may be introduced into the catalyst using nitrate salts. Additionally, magnesium may be introduced into the catalyst as an insoluble carbonate or hydroxide which upon heat treating results in an oxide. Phosphorus may be introduced in the catalyst as an alkaline metal salt, alkaline earth metal salt, the ammonium salt, or as phosphoric acid.

Required and/or optional alkali components of the catalyst composition (e.g., Rb, Li, Na, K, Cs, or mixtures thereof) may be introduced into the catalyst as an oxide or as a salt, which upon calcination will yield the oxide. In certain embodiments, salts such as nitrates which are readily available and easily soluble are used to incorporate such elements into the catalyst.

For the conversion of propylene, ammonia and oxygen to acrylonitrile, the inclusion of certain elements have been identified as being detrimental to obtaining a catalyst with improved acrylonitrile yields. For example, the inclusion of vanadium produces a catalyst composition that is more active in reacting the propylene feedstock and less selective to the desired products thereby producing more carbon oxides (COₓ) and less acrylonitrile. As such, in one embodiment, the catalyst composition is substantially free of vanadium. As used herein, “substantially free”, with respect to vanadium means having an atomic ratio with respect to molybdenum of less than 0.2:12.

Catalyst compositions described herein may be analyzed using X-ray diffraction techniques such as those described below in Example 3. In certain embodiments, catalyst compositions described herein exhibit certain X-ray diffraction (XRD) patterns, including peaks at a 2θ angle of about 23±0.3 degrees, about 28±0.3 degrees and/or about 26.5±0.3 degrees.

In one embodiment, the catalyst composition exhibits X-ray diffraction peaks at a 2θ angle of about 28±0.3 degrees and about 26.5±0.3 degrees, and the ratio of intensity of the most intense X-ray diffraction peak within a 2θ angle of about 28±0.3 degrees to the most intense X-ray diffraction peak within a 2θ angle of about 26.5±0.3 degrees is about 0.50 or less, about 0.40 or less, about 0.30 or less, about 0.20 or less, about 0.18 or less, about 0.16 or less, about 0.14 or less, about 0.12 or less, or about 0.1 or less. For example, from about 0.05 to about 0.5, from about 0.05 to about 0.4, from about 0.1 to about 0.4, from about 0.1 to about 0.3, or from about 0.1 to about 0.2.

In certain embodiments, the catalyst composition exhibits X-ray diffraction peaks at a 2θ angle of about 23±0.3 degrees and about 26.5±0.3 degrees, and the ratio of intensity of the most intense X-ray diffraction peak within a 2θ angle of about 23±0.3 degrees to the most intense X-ray diffraction peak within a 2θ angle of about 26.5±0.3 degrees is about 0.1 or greater, about 0.11 or greater, about 0.12 or greater, about 0.13 or greater, about 0.14 or greater, about 0.15 or greater, about 0.16 or greater, about 0.17 or greater, about 0.18 or greater, about 0.19 or greater, about 0.20 or greater, about 0.25 or greater, about 0.3 or greater, about 0.35 or greater, about 0.4 or greater, about 0.45 or greater, or about 0.5 or greater. For example, from about 0.1 to about 0.5, from about 0.11 to about 0.5, from about 0.12 to about 0.5, from about 0.13 to about 0.5, from about 0.14 to about 0.5, from about 0.15 to about 0.5, from about 0.15 to about 0.4, from about 0.15 to about 0.3, or from about 0.15 to about 0.2.

In certain embodiments, the catalyst composition exhibits X-ray diffraction peaks at a 2θ angle of about 23±0.3 degrees and about 28±0.3 degrees, and the ratio of intensity of the most intense X-ray diffraction peak within a 2θ angle of about 23±0.3 degrees to the most intense X-ray diffraction peak within a 2θ angle of about 28±0.3 degrees is about 0.5 or greater, about 0.6 or greater, about 0.7 or greater, about 0.8 or greater, about 0.9 or greater, about 1 or greater, about 1.2 or greater, about 1.4 or greater, about 1.6 or greater, about 1.8 or greater, about 2 or greater, about 2.2 or greater, about 2.4 or greater, about 2.6 or greater, about 2.8 or greater, or about 3 or greater. For example, from about 0.2 to about 3, from about 0.2 to about 2, from about 0.3 to about 3, or from about 0.3 to about 2. In another embodiment, the catalyst composition exhibits X-ray diffraction peaks at a 2θ angle of about 23±0.3 degrees and about 28±0.3 degrees, and the ratio of intensity of the most intense X-ray diffraction peak within a 2θ angle of about 23±0.3 degrees to the most intense X-ray diffraction peak within a 2θ angle of about 28=0.3 degrees is from about 0.5 to about 2, from about 1 to about 2, from about 1.2 to about 2, from about 1.4 to about 2, or from about 1.4 to about 1.8.

In certain other embodiments, the catalyst composition may be analyzed using X-ray diffraction (XRD) and a modified Rietveld analysis. In this aspect, crystallographic phases of a catalytic composition are analyzed using XRD analysis known in the art. A diffraction pattern of the catalytic composition is then analyzed with the modified Rietveld analysis described herein.

In accordance with the modified Rietveld analysis, a complete diffraction pattern is simulated through an ab initio calculation on the basis of the atomic structures of the individual phases from an assumed phase composition of the measuring sample. The correspondence between the simulated and measured diffraction pattern can then be effected through determination of covariance.

Rietveld analysis may be conducted using GSAS software as described in Larson et al., “General Structural Analysis System (GSAS)”, Los Alamos National Laboratory Report LAUR 86-784 (2004) and in Toby, “EXPGUI, A Graphical User Interface for GSAS”, J. Appl. Cryst., 34, 210-221 (2001), both of which are incorporated herein by reference. GSAS and EXPGUI are available at https://subversion.xor.aps.anl.gov/trac/EXPGUI/wiki.

The modified Rietveld model includes four phases which can be described as follows.

| Phase | Model Parameters | Refinement |
|---|---|---|
| β-MMoO₄ | Based on β-MMoO₄ | Unit cell and Fe |
| | Start from literature | occupancies |
| | structure: Sleight et al., | |
| | Inorg. Chem. 7, 1093-8 | |
| | (1968) | |
| Fe₂(MoO₄)₃ | Start from literature | |
| | structure: Chen, Mater. | |
| | Res. Bull., 14, 1583-90 | |
| | (1979) | |
| m-phase | Based on Ce₂(MoO₄)₃ | Refine unit |
| | Start from literature | cell and Ce |
| | structure: Brixner et al., J. | occupancies |
| | Solid State Chem., 5, 247-9 | |
| | (1972) | |
| t-phase | Based on NaBi(MoO4)2 | Refine unit cell |
| | Start from literature | |
| | structure: Waskowska et | |
| | al., Solid State Chem., 178, | |
| | 2218-24 (2005) | |

As used herein, “m-phase” refers to a component that is monoclinic scheelite like as determined by a modified Rietveld analysis as described herein.

As used herein, “t-phase” refers to a component that is tetragonal scheelite like as determined by modified Rietveld analysis described herein.

Starting atom coordinates are the same as reported in literature references. Starting lattice parameters are given here and differ slightly from the literature values. Thermal displacement parameters Uᵢₛₒ are given in units of Å².
β-FeMoO₄, structure described in Sleight et al., Inorg. Chem. 7, 1093-8 (1968), which is incorporated herein by reference.
space group C2/m, a=10.194 Å, b=9.229 Å, c=7.012 Å, β=107.08°.
Uᵢₛₒ 0.01 for Fe, 0.005 for Mo, 0.02 for O.
Starting Fe occupancies both 1.000.
Fe₂(MoO₄)₃, structure described in Chen, Mater. Res. Bull., 14, 1583-90 (1979), which is incorporated herein by reference.
space group P2₁/a, a=15.820 Å, b=9.347 Å, c=18.196 Å, β=125.60°.
Uᵢₛₒ 0.01 for Fe and Mo, 0.02 for O.
Ce₂(MoO₄)₃, structure described in Brixner et al., J. Solid State Chem., 5, 247-9 (1972), which is incorporated herein by reference.
space group C2/c, a=16.881 Å, b=11.825 Å, c=15.953 Å, β=108.73°.
Uᵢₛₒ 0.01 for Ce and Mo, 0.02 for O.
Starting Ce occupancies all 1.000.
NaBi(MoO₄)₂, structure described in Waskowska et al., Solid State Chem., 178, 2218-24 (2005), which is incorporated herein by reference.
space group I4₁/a, a=5.322 Å, c=11.851 Å.
Uᵢₛₒ 0.01 for Mo, 0.02 for Na, Bi, and O.
Background is modeled using either a 3-term cosine Fourier series or a 3-term shifted Chebyshev polynomial.

The amorphous component of the catalyst is modeled using seven Debye scattering terms with correction for thermal motion (diffuse scattering function 1 in GSAS). Each term is modeled as an Si—O vector with a thermal displacement parameter (U) of 0.05 Å². The Si—O distances of the seven terms are fixed at 1.55, 2.01, 2.53, 2.75, 3.49, 4.23, and 4.97 Å, and their amplitudes are optimized in the Rietveld fit.

The phases and parameters are introduced into the model gradually to ensure a stable refinement. At each step, 5-10 cycles of least-squares refinement are conducted to allow the model to settle down before the next components are introduced. A damping factor of 5 (i.e., 50%) on all parameters except the scale factors of the phases is used to reduce overshoots and oscillations. The procedure is as follows:
1. The starting model contains just the β-FeMoO₄ phase with its lattice parameters fixed and its profile Y (Lorentzian lattice strain) set to 75. Only the 3-term background function and the scale factor of the β-FeMoO₄ phase are varied.
2. The shift parameter (sample displacement) is added.
3. The lattice parameters of β-FeMoO₄ are allowed to vary.
4. The other three phases are added, all with fixed lattice parameters and profile X (Lorentzian Scherrer broadening) set to 20, and their scale factors are allowed to vary.
5. The 7 diffuse scattering terms are added and their amplitudes are allowed to vary.
6. Lattice parameters of the two scheelite-like phases are allowed to vary.
7. Profile Y of β-FeMoO₄ and profile X of the other three phases are allowed to vary.
8. Fe occupancies of the β-FeMoO₄ phase and Ce occupancies of the Ce₂(MoO₄)₃ phase are allowed to vary.
9. Least-squares refinement is continued until convergence, i.e., the sum of (shift/esd)² over all parameters is less than 0.01.

XRD peaks associated with the β-MMoO₄ phase (M=Ni, Mg, Fe), Fe₂(MoO₄)₃ phase, m-phase and t-phase are refined as described herein and the ratio of scheelite (m-phase+t-phase):β-MM004 is calculated based on the refined values. In certain embodiments, catalyst compositions of the present invention exhibit a significantly reduced scheelite:β-MMoO₄ ratio. For example, the calculated sheelite:β-MMoO₄ ratio following Rietveld refinement is no greater than about 0.3, no greater than about 0.25, no greater than about 0.2, no greater than about 0.15, no greater than about 0.1, no greater than about 0.09, no greater than about 0.08, no greater than about 0.07, no greater than about 0.06, no greater than about 0.05, no greater than about 0.04, no greater than about 0.03, no greater than about 0.02, or no greater than about 0.01. Catalyst exhibiting a significantly reduced scheelite:β-MMoO4 ratio following Rietveld refinement in combination with the compositional requirements, including a c (iron) to d (chromium) atomic ratio as disclosed above, provide for high conversion and desirable selectivity in the ammoxidation of an olefin, such as propylene to produce acrylonitrile.

The catalyst composition comprising oxides of molybdenum, bismuth, cerium, iron, chromium, at least one element of group A, at least one element of group B, and optionally at least one element of group C as described herein may be either supported or unsupported (i.e., the catalyst composition may further comprise a support). Suitable supports, for example, may be selected from the group consisting of silica, alumina, zirconium, titania, or mixtures thereof. A support typically serves as a binder for the catalyst, resulting in a harder and more attrition resistant catalyst and for that reason supported catalysts are typically employed when the ammoxidation reaction is conducted in a fluidized bed reactor. For commercial applications, an appropriate blend of both the catalytic oxides described above and the support is selected to obtain an acceptable activity and hardness (attrition resistance) for the catalyst. Increase in the proportion of the catalytic oxides increases the activity of the catalyst, but decreases the hardness of the catalyst. Typically, when employed, the support comprises from about 30 wt. % to about 70 wt. %, from about 35 wt. % to about 70 wt. %, from about 40 wt. % to about 70 wt. %, from about 40 wt. % to about 65 wt. %, from about 40 wt. % to about 60 wt. %, from about 40 wt. % to about 55 wt. %, or from about 45 wt. % to about 55 wt. % of the catalyst composition. In certain embodiments, the support materials may contain one or more promoter elements (e.g., a silica sol containing sodium (Na)), and such promoter elements may be incorporated into the catalyst composition via the support material.

In one embodiment the catalyst is supported using a silica sol. If the average colloidal particle diameter of the silica sol is too small, the surface area of the manufactured catalyst will be increased and the catalyst will exhibit reduced selectivity. If the colloidal particle diameter is too large, the manufactured catalyst will have poor anti-abrasion strength. Therefore, in certain embodiments, the average colloidal particle diameter of the silica sol is between about 8 nm and about 50 nm.

The catalysts of the present invention may be prepared by any of the numerous methods of catalyst preparation that are known to those of skill in the art. For example, the catalyst may be manufactured by co-precipitating the various ingredients. The co-precipitating mass may then be dried and ground to an appropriate size. Alternatively, the co-precipitated material may be slurried and spray dried in accordance with conventional techniques. The catalyst may be extruded as pellets or formed into spears in oil as is well known in the art. For particular procedures for manufacturing the catalyst, see U.S. Pat. Nos. 5,093,299; 4,863,891 and 4,766,232, herein incorporated by reference. In one embodiment, the catalyst components may be mixed with a support in the form of the slurry followed by drying. In another embodiment, the catalyst components may be impregnated on silica or other supports.

The catalyst composition is prepared by mixing compounds of the requisite elements in appropriate molar quantities for the desired catalytic oxide composition. Typically, for a silica supported catalyst, an aqueous solution of ammonium heptamolybdate is mixed with a silica sol to which a slurry containing the compounds (e.g., nitrate salts) of the other elements is added. The solid material is then dried, denitrified and calcined. The catalyst may be spray-dried at a temperature of from about 110° C. to about 350° C., from about 110° C. to about 250° C., or from about 110° C. to about 180° C. The denitrification temperature may range from about 100° C. to about 500° C., or from about 250° C. to about 450° C. Calcination may take place at a temperature of between about 300° C. and about 700° C., or between about 350° C. and about 650° C. The composition of the resulting catalytic oxide can be determined by means known in the art such as Inductively Coupled Plasma (ICP) analysis.

The catalysts described herein are useful in ammoxidation processes for the conversion of an unsaturated hydrocarbon to the corresponding unsaturated nitrile by reacting in the vapor phase at an elevated temperature and pressure the olefin with a molecular oxygen containing gas and ammonia in the presence of the catalyst. For example, conversion of an olefin selected from the group consisting of propylene, isobutylene or mixtures thereof to acrylonitrile, methacrylonitrile and mixtures thereof, respectively, by reacting in the vapor phase at an elevated temperature and pressure the olefin with a molecular oxygen containing gas and ammonia in the presence of the catalyst.

In certain embodiments, the ammoxidation reaction is performed in a fluidized bed reactor. For example, the reactor design set forth in U.S. Pat. No. 3,230,246, herein incorporated by reference, is suitable. In another embodiment, other types of known reactors, such as transport line reactors, may be used for the ammoxidation reaction.

Conditions for the ammoxidation reaction are described, for example, in U.S. Pat. Nos. 5,093,299; 4,863,891; 4,767,878 and 4,503,001; incorporated herein by reference. Typically, the ammoxidation process is performed by contacting propylene or isobutylene in the presence of ammonia and oxygen with a fluidized bed catalyst at an elevated temperature to produce the acrylonitrile or methacrylonitrile.

Any suitable source of oxygen may be employed. For economic reasons, however, it may be desirable to utilize air as the source of oxygen. In certain embodiments, the molar ratio of the oxygen to olefin in the feed is from about 0.5:1 to about 4:1, or from about 1:1 to about 3:1. In certain specific embodiments, the olefin comprises propylene, the source of oxygen comprises air, and the molar ratio of air to propylene is from about 5:1 to about 20:1, from about 5:1 to about 15:1, from about 6:1 to about 12:1, from about 7:1 to about 15:1, from about 8:1 to about 15:1, from about 8:1 to about 14:1, from about 8:1 to about 13:1, or from about 8:1 to about 12:1.

Generally, for economic reasons, the molar ratio of ammonia to olefin in the feed in the reaction is a ratio of about 2:1 or less. For example, in certain embodiments, the molar ratio of ammonia to olefin in the feed in the reaction may vary from between 0.5:1 to 2:1. For example, in one embodiment, the molar ratio of ammonia to olefin in the feed in the reaction is from about 0.5:1 to about 2:1, from about 0.5:1 to about 1.5:1, from about 0.5:1 to about 1.4:1, from about 0.5:1 to about 1.3:1, from about 0.5:1 to about 1.2:1, from about 0.5:1 to about 1.1:1, from about 0.5:1 to about 1:1, or from about 0.75:1 to about 1:1.

It has been discovered that, in certain embodiments, the catalyst described herein provide high yields of acrylonitrile at relatively low ammonia to propylene feed ratios. For example, in one embodiment, the olefin comprises propylene and the molar ratio of ammonia to propylene is from about 0.9:1 to about 1.3:1, from about 0.9:1 to about 1.2:1, from about 0.9:1 to about 1.1:1, from about 1:1 to about 1.1:1, or from about 1:1 to about 1.05:1. These “low ammonia conditions” help to reduce unreacted ammonia in the reactor effluent, a condition known as “ammonia breakthrough”, which subsequently helps to reduce process wastes. Specifically, unreacted ammonia must be removed from the reactor effluent prior to the recovery of the acrylonitrile. Unreacted ammonia is typically removed by contacting the reactor effluent with sulfuric acid to ammonium acrylate, which in both cases results in a process waste stream to be treated and/or disposed. Further, as ammonia is a relatively expensive reagent, increasingly efficient utilization of ammonia further reduces the production costs.

In one embodiment for the production of acrylonitrile from propylene, the ammonia to propylene ratio is from about 0.9:1 to about 1.3:1 and the air to propylene ratio is from about 8.0:1 to about 12.0:1.

The reaction may be carried out at a temperature of from about 260° C. to about 600° C., from about 310° C. to about 500° C., or from about 350° C. to about 480° C. The contact time, although not critical, is generally from about 0.1 to about 50 seconds. In certain embodiment, the contact time is from about 1 to about 15 seconds.

The products of the reaction may be recovered and purified by any known method. One such method involves scrubbing the effluent gases from the reactor with cold water or an appropriate solvent to remove the products of the reaction and then purifying the reaction product by distillation.

Catalysts of the present invention have been found to surprisingly contribute to an improved olefin conversion. For example, by utilizing the catalyst of the present invention in an olefin ammoxidation process wherein the olefin comprises propylene, the olefin conversion is about 85% or greater, about 86% or greater, about 87% or greater, about 88% or greater, about 89% or greater, about 90% or greater, about 91% or greater, about 92% or greater, about 93% or greater, about 94% or greater, about 95% or greater, about 96% or greater, about 97% or greater, about 98% or greater, about 99% or greater, about 99.1% or greater, about 99.2% or greater, about 99.3% or greater, about 99.7% or greater, about 99.8% or greater, about 99.6% or greater, about 99.7% or greater, about 99.8% or greater, or about 99.9% or greater, and typically from about 98% to about 99%.

Catalysts of the present invention have been found, in some embodiments, to surprisingly contribute to an improved nitrile yield. For example, by utilizing the catalyst of the present invention in an olefin ammoxidation process wherein the olefin comprises propylene, the nitrile yield is about 70% or greater, about 72% or greater, about 74% or greater, about 76% or greater, about 78% or greater, about 80% or greater, about 82% or greater, about 84% or greater, about 86% or greater, about 88% or greater, or about 90% or greater.

Although the catalysts are described herein for the ammoxidation of propylene to acrylonitrile, the catalyst may also be used for the oxidation of propylene to acrylic acid. Such processes are typically two stage processes, wherein propylene is converted in the presence of a catalyst to primarily acrolein in the first stage and the acrolein is converted in the presence of a catalyst to primarily acrylic acid in the second stage. The catalysts described herein are suitable for use in the first stage for the oxidation of propylene to acrolein.

### EXAMPLES

In order to illustrate the instant invention, catalysts of the instant invention as well as similar catalysts omitting one or more of the required elements and/or comprising the elements in different proportions, were prepared and then evaluated under similar reaction conditions. These examples are provided for illustrative purposes only.

### Example 1—Catalyst Preparation

### Sample 1. A Catalyst of the Formula 50%

Ni_{3.85}Mg_{2.89}Fe_{0.843}Rb_{0.13}Cr_{0.082}Bi_{1.35}Ce_{0.67}Mo₁₂O_{47.57}+50 wt % SiO₂ was prepared as follows.

Metal nitrates, in the following order, Fe(NO₃)₃·9H₂O (32.15 g), Ni(NO₃)₂·6H₂O (105.83 g), Mg(NO₃)₂·6H₂O (69.98 g), Bi(NO₃)₃·5H₂O (61.77 g), Cr(NO₃)_{3·}9H₂O (3.08 g), RbNO₃ (1.81 g), (NH₄)₂Ce(NO₃)₆ (69.81 g of a 50% solution) were dissolved in water (30.51 g) at around 55° C. to form a mixed metal nitrates solution. Ammonium heptamolybdate (AHM) (200.07 g) was dissolved in 220.08 g of distilled water at around 65° C. Silica (746.27 of a 33.5% SiO₂ sol) was then added, followed by the mixed metal nitrates solution. The resulted slurry was then spray dried. The obtained material was denitrified at 290° C. for 3 hours and 425° C. for 3 hours, and then calcined at 580° C. for 3 hours, in air.

### Sample 2. A Catalyst of Formula 50%

Ni_{3.43}Mg_{2.57}Fe_{1.50}Rb_{0.13}Cr_{0.36}Bi_{0.60}Ce_{1.20}Mo₁₂O_{48.16}+50 wt % SiO₂ was prepared as follows. Metal nitrates, in the following order, Fe(NO₃)3·9H₂O (58.43 g), Ni(NO₃)₂·6H₂O (96.15 g), Mg(NO₃)₂·6H₂O (63.56 g), Bi(NO₃)₃·5H₂O (28.05 g), Cr(NO₃)₃·9H₂O (13.99 g), RbNO₃ (1.85 g), (NH₄)₂Ce(NO₃)₆ (126.91 g of a 50% solution) were dissolved in water (29.11 g) at around 55° C. to form a mixed metal nitrates solution. Ammonium heptamolybdate (AHM) (204.29 g) was dissolved in 224.72 g of distilled water at around 65° C. Silica (746.27 of a 33.5% SiO2 sol) was then added, followed by the mixed metal nitrates solution. The resulted slurry was then spray dried. The obtained material was denitrified at 290° C. for 3 hours and 425° C. for 3 hours and then calcined at 580° C. for 3 hours, in air.

### Sample 3. A Catalyst of Formula 50%

Ni_{3.89} Mg_{2.92}Fe_{1.75}Rb_{0.19}Cr_{0.05}Bi_{0.19}Ce_{1.22}Mo₁₂O_{48.32}+50 wt % SiO₂ was prepared as follows. Metal nitrates, in the following order, Fe(NO₃)₃·9H₂O (69.29 g), Ni(NO₃)₂·6H₂O (110.82 g), Mg(NO₃)₂·6H₂O (73.29 g), Bi(NO₃)₃·5H₂O (9.24 g), Cr(NO₃)₃.9H₂O (1.91 g), RbNO₃ (2.7 g), (NH₄)₂Ce(NO₃)₆ (132.88 g of a 50% solution) were dissolved in water (29.69 g) at around 55° C. to form a mixed metal nitrates solution. Ammonium heptamolybdate (AHM) (207.69 g) was dissolved in 228.45 g of distilled water at around 65° C. Silica (746.27 of a 33.5% SiO2 sol) was then added, followed by the mixed metal nitrates solution. The resulted slurry was then spray dried. The obtained material was denitrified at 290° C. for 3 hours and 425° C. for 3 hours, and then calcined at 580° C. for 3 hours, in air.

### Sample 4. A Catalyst of Formula 50%

Ni_{3.89} Mg_{2.92}Fe_{0.36}Rb_{0.19}Cr_{0.05}Bi_{0.36}Ce_{2.43}Mo₁₂O_{48.92}+50 wt % SiO₂ was prepared as follows. Metal nitrates, in the following order, Fe(NO₃)₃·9H₂O (13.69 g), Ni(NO₃)₂·6H₂O (105.13 g), Mg(NO₃)₂·6H₂O (69.52 g), Bi(NO₃)₃·5H₂O (16.44 g), Cr(NO₃)₃·9H₂O (1.81 g), RbNO₃ (2.56 g), (NH₄)₂Ce(NO₃)₆ (252.09 g of a 50% solution) were dissolved in water (23.24 g) at around 55° C. to form a mixed metal nitrates solution. Ammonium heptamolybdate (AHM) (197.01 g) was dissolved in 216.72 g of distilled water at around 65° C. Silica (746.27 of a 33.5% SiO2 sol) was then added, followed by the mixed metal nitrates solution. The resulted slurry was then spray dried. The obtained material was denitrified at 290° C. for 3 hours and 425° C. for 3 hours, and then calcined at 580° C. for 3 hours, in air.

### Comparative Catalysts

### Comparative Example a. A Catalyst of the Formula 50 wt %

Ni_{1.81} Mg_{1.36}Fe_{2.64}Rb_{0.13}Cr_{0.13}Bi_{1.05}Ce_{2.11}Mo₁₂O_{49.18}+50 wt % SiO₂ was prepared as follows. Metal nitrates, in the following order, Fe(NO₃)₃·9H₂O (96.57 g), Ni(NO₃)₂·6H₂O (47.67 g), Mg(NO₃)₂·6H₂O (31.53 g), Bi(NO₃)₃·5H₂O (46.36 g), Cr(NO₃)₃·9H₂O (4.63 g), RbNO₃ (1.74 g), (NH₄)₂Ce(NO₃)₆ (209.69 g of a 50% solution) were dissolved in water (25.39 g) at around 55° C. to form a mixed metal nitrates solution. Ammonium heptamolybdate (AHM) (192.03 g) was dissolved in 211.23 g of distilled water at around 65° C. Silica (746.27 of a 33.5% SiO2 sol) was then added, followed by the mixed metal nitrates solution. The resulted slurry was then spray dried. The obtained material was denitrified at 290° C. for 3 hours and 425° C. for 3 hours, and then calcined at 580° C. for 3 hours, in air.

Several other comparative catalysts were prepared using techniques and procedure described in Example 1 of U.S. Pat. No. 7,071,140. In the catalyst of Comparative Examples B-E, one or more of chromium, cerium or rubidium was omitted from the preparation. In Comparative Examples D and E, cesium (CsNO₃) and potassium (KNO₃) was substituted for rubidium, respectively.

### Comparative Example B. 50 wt %

Ni_{5.0} Mg_{2.0}Fe_{1.8}Bi_{0.45}Ce_{0.9}Rb_{0.15}Mo₁₂O_{48.25}+50 wt % SiO₂.

### Comparative Example C. 50 wt %

Ni_{5.0} Mg_{2.0}Fe_{1.8}Bi_{0.45}Cr_{0.1}Rb_{0.15}Mo₁₂O_{46.6}+50 wt % SiO₂.

### Comparative Example D. 50 wt %

Ni_{5.0} Mg_{2.0}Fe_{1.8}Bi_{0.45}Ce_{0.9}Cr_{0.1}K_{0.15}Mo₁₂O_{48.4}+50 wt % SiO₂.

### Comparative Example E. 50 wt %

Ni_{5.0} Mg_{2.0}Fe_{1.8}Bi_{0.45}Ce_{0.9}Cr_{0.1}Cs_{0.15}Mo₁₂O_{48.4}+50 wt % SiO₂.

### Comparative Example F. 50 wt %

Ni_{5.0} Mg_{2.0}Fe_{1.8}Bi_{0.45}Ce_{0.9}Cr_{0.1}Rb_{0.15}Mn_{1.0}Mo₁₂O_{49.4}+50 wt % SiO₂. This catalyst added manganese, Mn(NO₃)₂ (32.699 g of a 51.1% solution), to the catalyst preparation.

### Comparative Example G. 50 wt %

Ni_{5.0} Mg_{2.0}Fe_{1.8}Bi_{0.45}Ce_{0.9}Cr_{0.1}Rb_{0.15}Pd_{0.1}Mo₁₂O_{48.5}+50 wt % SiO₂. This catalyst added a noble metal, palladium, Pd(NO₃)₂ (2.2 g) to the catalyst preparation.

### Comparative Example H. 50 wt %

Ni_{5.0} Mg_{2.0}Fe_{1.8}Bi_{0.45}Ce_{0.9}Cr_{0.1}Rb_{0.15} V_{0.5}Mo₁₂O_{49.65}+50 wt % SiO₂. This catalyst added vanadium, NH₄VO₃ (5.514 g) to the catalyst preparation.

### Comparative Example I. 50 wt %

Ni_{5.0}Mg_{2.0}Fe_{1.8}Bi_{0.45}Ce_{0.15}Cr_{0.3}Rb_{0.15}Mo₁₂O_{47.2}+50 wt % SiO₂. This catalyst was prepared wherein, on an atomic basis, the molar quantity of cerium plus the molar quantity of chromium equals the molar quantity of bismuth. The recipe of this catalyst is as follows: Fe(NO₃)₃·9H₂O (72.939 g), Ni(NO₃)₂·6H₂O (145.83 g), Mg(NO₃)₂·6H₂O (51.434 g), Bi(NO₃)₃·5H₂O (21.894 g), RbNO₃ (2.219 g), and (NH₄)₂Ce(NO₃)₆ (16.496 g of a 50% solution) were dissolved in water at ˜70° C. in a 1000 ml beaker. Ammonium heptamolybdate (AHM) (212.504 g) was dissolved in 310 ml of distilled water. To this solution CrO₃ (3.009 g) dissolved in a 20 ml water was added. Then the silica (871.08 g of a 28.75% SiO₂ sol) was added followed by the metal nitrates.

### Comparative Example J. 50 wt %

Ni_{5.0} Mg_{2.0}Fe_{1.8}Bi_{0.45}Ce_{0.1}Cr_{0.1}Rb_{0.15}Mo₁₂O_{46.8}+50 wt % SiO₂. This catalyst was prepared wherein, on an atomic basis, the quantity of cerium plus the quantity of chromium is less than the quantity of bismuth. The recipe of this catalyst is as follows: Fe(NO₃)₃·9H₂O (73.642 g), Ni(NO₃)₂·6H₂O (147.236 g), Mg(NO₃)₂·6H₂O (51.93 g), Bi(NO₃)₃·5H₂O (22.105 g), RbNO₃ (2.24 g), (NH₄)₂Ce(NO₃)₆ (11.104 g of a 50% solution) were dissolved in water at ˜70° C. in a 1000 ml beaker. Ammonium heptamolybdate (AHM) (214.553 g) was dissolved in 310 ml of distilled water. To this solution CrO₃ (1.013 g) dissolved in a 20 ml water was added. Then the silica (871.08 g of a 28.75% SiO₂ sol) was added followed by the metal nitrates.

### Comparative Example K. 50 wt %

Ni_{5.0} Mg_{2.0}Fe_{1.8}Bi_{2.0}Ce_{0.9}Cr_{0.1}Rb_{0.15}Mo₁₂O_{46.8}+50 wt % SiO₂. This catalyst was prepared wherein, on an atomic basis, the quantity of cerium plus the quantity of chromium is less than the quantity of bismuth. The recipe of this catalyst is as follows: Fe(NO₃)₃·9H₂O (61.264 g), Ni(NO₃)₂·6H₂O (122.488 g), Mg(NO₃)₂·6H₂O (43.201 g), Bi(NO₃)₃·5H₂O (81.732 g), RbNO₃ (1.863 g), (NH)₂Ce(NO₃)₆ (83.136 g of a 50% solution) were dissolved in water at ˜70° C. in a 1000 ml beaker. Ammonium heptamolybdate (AHM) (178.49 g) was dissolved in 310 ml of distilled water. To this solution CrO₃ (0.843 g) dissolved in a 20 ml water was added. Then the silica (871.08 g of a 28.75% SiO₂ sol) was added followed by the metal nitrates.

ICP analysis of the resulting materials was conducted and it was confirmed that the catalysts comprised the formulae set forth above.

### Example 2—Catalyst Testing

Catalyst Samples 1-4 and Comparative Catalysts were tested in a 40 cc fluid bed reactor. Propylene was feed into the reactor at a rate of 0.06 WWH (i.e., weight of propylene/weight of catalyst/hour). Pressure inside the reactor was maintained at 10 psig. Reaction temperature was 430° C. After a stabilization period of ˜20 hours samples of reaction products were collected. Reactor effluent was collected in bubble-type scrubbers containing cold HCl solution. Off-gas rate was measured with soap film meter, and the off-gas composition was determined at the end of the run with the aid of gas chromatograph fitted with a split column gas analyzer. At the end of the recovery run, the entire scrubber liquid was diluted to approximately 200 gms with distilled water. A weighted amount of 2-Butanone was used as internal standard in a ˜50 grams aliquot of the dilute solution. A 2 μl sample was analyzed in a GC fitted with a flame ionization detector and a Carbowax column. The amount of NH₃ was determined by titrating the free HCl excess with NaOH solution. The results are set forth below.

TABLE 1

| | | Total | Conv. | Sel. |
|---|---|---|---|---|
| | | C3 | to | to |
| Catalyst | Catalyst Composition | Conv. | AN | AN |
| Sample | Ni_{3.85}Mg_{2.89}Fe_{0.843}Rb_{0.13}Cr_{0.082}Bi_{1.35}Ce_{0.67}Mo₁₂O_{47.57} | 99.25 | 82.06 | 82.68 |
| 1 | | | | |
| Sample | Ni_{3.43}Mg_{2.57}Fe_{1.50}Rb_{0.13}Cr_{0.36}Bi_{0.60}Ce_{1.20}Mo₁₂O_{48.16} | 98.8 | 82.9 | 84 |
| 2 | | | | |
| Sample | Ni_{3.89}Mg_{2.92}Fe_{1.75}Rb_{0.19}Cr_{0.05}Bi_{0.19}Ce_{1.22}Mo₁₂O_{48.32} | 99.2 | 82.3 | 82.9 |
| 3 | | | | |
| Sample | Ni_{3.89}Mg_{2.92}Fe_{0.36}Rb_{0.19}Cr_{0.05}Bi_{0.36}Ce_{2.43}Mo₁₂O_{48.92} | 89.9 | 79.7 | 80.7 |
| 4 | | | | |
| A | Ni_{1.81}Mg_{1.36}Fe_{2.64}Rb_{0.13}Cr_{0.13}Bi_{1.05}Ce_{2.11}Mo₁₂O_{49.18} | 96.8 | 76.2 | 78.8 |
| B | Ni_{5.0}Mg_{2.0}Fe_{1.8}Bi_{0.45}Ce_{0.9}Rb_{0.15}Mo₁₂O_{48.25} | 99.4 | 79.3 | 79.9 |
| C | Ni_{5.0}Mg_{2.0}Fe_{1.8}Bi_{0.45}Cr_{0.1}Rb_{0.15}Mo₁₂O_{46.6} | 91.2 | 75.8 | 83.1 |
| D | Ni_{5.0}Mg_{2.0}Fe_{1.8}Bi_{0.45}Ce_{0.9}Cr_{0.1}K_{0.15}Mo₁₂O_{48.4} | 99.7 | 77.6 | 77.8 |
| E | Ni_{5.0}Mg_{2.0}Fe_{1.8}Bi_{0.45}Ce_{0.9}Cr_{0.1}Cs_{0.15}Mo₁₂O_{48.4} | 96.8 | 69.6 | 72.0 |
| F | Ni_{5.0}Mg_{2.0}Fe_{1.8}Bi_{0.45}Ce_{0.9}Cr_{0.1}Rb_{0.15}Mn_{1.0}Mo₁₂O_{49.4} | 97.3 | 78.0 | 78.6 |
| G | Ni_{5.0}Mg_{2.0}Fe_{1.8}Bi_{0.45}Ce_{0.9}Cr_{0.1}Rb_{0.15}Pd_{0.1}Mo₁₂O_{48.5} | 99.3 | 78.7 | 81.4 |
| H | Ni_{5.0}Mg_{2.0}Fe_{1.8}Bi_{0.45}Ce_{0.9}Cr_{0.1}Rb_{0.15}V_{0.5}Mo₁₂O_{49.65} | 96.4 | 76.8 | 79.7 |
| I | Ni_{5.0}Mg_{2.0}Fe_{1.8}Bi_{0.45}Ce_{0.15}Cr_{0.3}Rb_{0.15}Mo₁₂O_{47.2} | 93.5 | 77.9 | 83.3 |
| J | Ni_{5.0}Mg_{2.0}Fe_{1.8}Bi_{0.45}Ce_{0.1}Cr_{0.1}Rb_{0.15}Mo₁₂O_{46.8} | 94.9 | 74.4 | 78.5 |
| K | Ni_{5.0}Mg_{2.0}Fe_{1.8}Bi_{2.0}Ce_{0.9}Cr_{0.1}Rb_{0.15}Mo₁₂O_{46.8} | 97.7 | 78.8 | 80.7 |

Notes:
1. The test catalyst compositions contained 50% catalyst oxides and 50% SiO₂ by weight.
2. “Total C3 Conv.” is the mole percent per pass conversion of propylene to all products.
3. “Conv. To AN” is the mole percent per pass conversion of propylene to acrylonitrile.
4. “Sel. to AN” is the ratio of moles of acrylonitrile produced to moles of propylene converted expressed in percent.

### Example 3—X-Ray Diffraction Analysis

Fresh catalyst samples were analyzed as received with no grinding. The sample powders are added to a zero-background cell to make a flat and densely packed sample for XRD analysis. Typical conditions for a Panalytical Empyrean Series 3 diffractometer were as follows:

### Sample Spinning

Cu Kα radiation (wavelength Kα1 1.5406 Å)
X-ray generator 45 KV, 40 mA
Incident beam optics:
   Divergence slit: Fixed slit ⅛°
   Antiscattering slit: Fixed slit ½°
Diffracted beam optics:
   Soller slit: Soller slit 0.02 rad
   Antiscattering slit: AS slit 7.5 mm
Scan range 5°-100°
Step size 0.013°
Total scan time 4:00 (240 min)

Catalyst Sample 2 and Comparative Example A were also evaluated for X-ray diffraction peaks and further refined using Rietveld refinement as detailed herein. The results are reported below in Tables 2 and 3.

“23°:28°” corresponds to the ratio of intensity of the most intense X-ray diffraction peak within a 2θ angle of about 23±0.3 degrees to the most intense X-ray diffraction peak within a 2θ angle of about 28±0.3 degrees.

TABLE 2

| Catalyst | Catalyst Composition | 23°:28° |
|---|---|---|
| Sam- | Ni_{3.43}Mg_{2.57}Fe_{1.50}Rb_{0.13}Cr_{0.36}Bi_{0.60}Ce_{1.20}Mo₁₂O_{48.16} | 1.473 |
| ple 2 | | |
| A | Ni_{1.81}Mg_{1.36}Fe_{2.64}Rb_{0.13}Cr_{0.13}Bi_{1.05}Ce_{2.11}Mo₁₂O_{49.18} | 0.548 |

Modified XRD Rietveld refinement was undertaken in accordance with the procedure described above. Peaks associated with the β-MMoO₄ phase (M=Ni, Mg, Fe) phase, Fe₂(MoO₄)₃ phase, m-phase and t-phase were refined and the ratio of scheelite (m-phase+t-phase):β-MMoO₄ was calculated. The results are reported below in Table 3.

TABLE 3

| | | β- | | | | | |
|---|---|---|---|---|---|---|---|
| | | MMoO₄ | | | | Scheelite | |
| | | (M = Ni, | | | | (m-phase + | Scheelite: β- |
| Catalyst | Catalyst Composition | Mg, Fe) | Fe₂(MoO₄)₃ | m-phase | t-phase | t-phase) | MMoO₄ |
| Sample 1 | Ni_{3.85}Mg_{2.89}Fe_{0.843}Rb_{0.13}Cr_{0.082}Bi_{1.35}Ce_{0.67}Mo₁₂O_{47.57} | 76.1% | 6.7% | 10.8% | 6.4% | 17.2% | 0.23 |
| Sample 2 | Ni_{3.43}Mg_{2.57}Fe_{1.50}Rb_{0.13}Cr_{0.36}Bi_{0.60}Ce_{1.20}Mo₁₂O_{48.16} | 68.7% | 28.9% | 2.4% | 0% | 2.4% | 0.035 |
| Sample 3 | Ni_{3.89}Mg_{2.92}Fe_{1.75}Rb_{0.19}Cr_{0.05}Bi_{0.19}Ce_{1.22}Mo₁₂O_{48.32} | 76.7% | 22.4% | 1.0% | 0% | 1.0% | 0.013 |
| Sample 4 | Ni_{3.89}Mg_{2.92}Fe_{0.36}Rb_{0.19}Cr_{0.05}Bi_{0.36}Ce_{2.43}Mo₁₂O_{48.92} | 81.0% | 0% | 4.0% | 15.1% | 19.1% | 0.24 |
| A | Ni_{1.81}Mg_{1.36}Fe_{2.64}Rb_{0.13}Cr_{0.13}Bi_{1.05}Ce_{2.11}Mo₁₂O_{49.18} | 29.2% | 37.9% | 19.9% | 13.00% | 32.9% | 1.13 |

From the elemental composition of the active phase, such as that given for Catalyst Sample 2 and Comparative Example A in Table 3, a representative percentage of m-phase based on the amount of Ce in the active phase as a percentage of the total active phase can be calculated. For cases where the stoichiometric amount of Bi in the active phase is greater than the stoichiometric amount of Ce in the active phase, a representative percentage of m-phase may be calculated assuming that all Ce is in the m-phase and the m-phase is described by the formula CeBi(MoO₄)₃. For cases where the stoichiometric amount of Ce in the active phase is greater than the stoichiometric amount of Bi in the active phase, a representative percentage of m-phase may be calculated using the formula CeₓBi₍₂₋ₓ₎(MoO₄)₃, where x is (2 n_{Ce}/(n_{Bi}+n_{Ce})). Based on the known active phase composition, known atomic weights of the various components, and the appropriate m-phase formula as described above, a representative weight percent of m-phase can be determined.

As shown in Table 4, the ratio of measured m-phase as measured by Rietveld analysis to the calculated representative m-phase is much lower in the catalyst compositions of the present invention (Sample 2) as compared to Comparative Example A.

TABLE 4

| | % Representative | % Measured | |
|---|---|---|---|
| | m-phase in | m-phase via | Ratio |
| Catalyst | active phase | Rietveld | Measured:Representative |
| Sample 2 | 34.58% | 2.40% | 0.06940428 |
| A | 34.48% | 19.90% | 0.577146172 |

Typically, in some embodiments, the ratio of measured m-phase as determined by Rietveld analysis to the calculated representative m-phase is no greater than about 0.1, no greater than about 0.09, no greater than about 0.08, no greater than about 0.07, no greater than about 0.06, no greater than about 0.05, no greater than about 0.04, no greater than about 0.03, or no greater than about 0.02.

The catalyst composition of the instant invention is unique in that it comprises a complex of catalytic oxides of molybdenum, bismuth, cerium, iron, chromium, at least one element of group A, at least one element of group B, and optionally at least one element of group C in specific proportions. This combination of elements in the relative proportions described herein have not previously utilized in a single ammoxidation catalyst formulation. As illustrated in Table 1, for the ammoxidation of propylene to acrylonitrile, a catalyst of the instant invention has exhibited better performance than catalysts comprising similar combinations of elements. More specifically, catalysts of the present invention exhibited a combination of higher overall conversion of propylene and high selectivity to acrylonitrile compared to similar catalysts.

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

While the foregoing description and the above embodiments are typical for the practice of the instant invention, it is evident that many alternatives, modifications, and variations will be apparent to those skilled in the art in light of this description. Accordingly, it is intended that all matter contained in the above description shall be interpreted as illustrative and not in a limiting sense and that all such alternatives, modifications and variations are embraced by and fall within the spirit and broad scope of the appended claims.

1. A catalyst composition comprising a complex of catalytic oxides comprising molybdenum, bismuth, cerium, iron, chromium, at least one element of group A, at least one element of group B, and optionally at least one element of group C,
wherein group A consists of sodium, potassium, rubidium, and cesium;
wherein group B consists of nickel, cobalt, manganese, zinc, magnesium, calcium, strontium, cadmium, and barium;
wherein group C consists of silver, gold, ruthenium, rhodium, palladium, osmium, iridium, platinum, and mercury; and
wherein the relative ratios of these elements are represented by Formula (1): 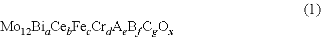
wherein:
a is from about 0.05 to about 4;
b is from about 0.3 to about 3;
c is from about 0.01 to about 4;
d is from about 0.01 to about 2;
e is from about 0.01 to about 2;
f is from about 0.01 to about 10;
g is from about 0 to about 0.2;
x is a number determined by the valence requirement of the other elements present; and
wherein c/d is from about 2 to 17.

2. The catalyst composition of claim 1, wherein c/d is from about 7 to about 12 from about 7 to about 11.5, from about 7 to about 11, from about 7 to about 10.5, from about 7.5 to about 10.5, from about 8 to about 10.5, or from about 8 to about 10.

3. The catalyst composition of claim 1, wherein c/d is from about 2 to 16, from about 2 to about 14, from about 2 to about 12, from about 2 to about 10, from about 2 to about 8, or from about 2 to about 6.

4. The catalyst composition of any one of claims 1 to 3, wherein the catalyst composition exhibits a scheelite (m-phase+t-phase):β-MMoO₄ ratio of no greater than about 0.1, no greater than about 0.09, no greater than about 0.08, no greater than about 0.07, no greater than about 0.06, no greater than about 0.05, no greater than about 0.04, no greater than about 0.03, no greater than about 0.02, or no greater than about 0.01, wherein amounts of m-phase, t-phase and β-MMoO₄ phase are determined using X-ray diffraction and a modified Rietveld analysis model.

5. A catalyst composition comprising a complex of catalytic oxides comprising molybdenum, bismuth, cerium, iron, chromium, at least one element of group A, at least one element of group B, and optionally at least one element of group C,
wherein group A consists of sodium, potassium, rubidium, and cesium;
wherein group B consists of nickel, cobalt, manganese, zinc, magnesium, calcium, strontium, cadmium, and barium;
wherein group C consists of silver, gold, ruthenium, rhodium, palladium, osmium, iridium, platinum, and mercury; and
wherein the relative ratios of these elements are represented by Formula (1): 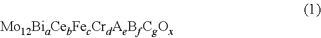
wherein:
a is from about 0.05 to about 4;
b is from about 0.01 to about 3;
c is from about 0.01 to about 4;
d is from about 0.01 to about 2;
e is from about 0.01 to about 2;
f is from about 0.01 to about 10;
g is from about 0 to about 0.2;
x is a number determined by the valence requirement of the other elements present; and
wherein c/d is from about 2 to 36 and the catalyst composition exhibits a scheelite (m-phase+t-phase):β-MMoO₄ ratio of no greater than about 0.3, wherein amounts of m-phase, t-phase and β-MMoO₄ phase are determined using X-ray diffraction and a modified Rietveld analysis model.

6. The catalyst composition of claim 5, wherein the catalyst composition exhibits a scheelite (m-phase+t-phase):β-MMoO₄ ratio of no greater than about 0.25, no greater than about 0.2, no greater than about 0.15, no greater than about 0.1, no greater than about 0.09, no greater than about 0.08, no greater than about 0.07, no greater than about 0.06, no greater than about 0.05, no greater than about 0.04, no greater than about 0.03, no greater than about 0.02, or no greater than about 0.01, wherein amounts of m-phase, t-phase and β-MMoO₄ phase are determined using X-ray diffraction and a modified Rietveld analysis model.

7. The catalyst composition of claim 5 or claim 6, wherein c/d is from about 2 to about 32, from about 2 to about 28, from about 2 to about 24, from about 2 to about 20, from about 4 to about 36, from about 4 to about 32, from about 4 to about 28, from about 4 to about 24, from about 4 to about 20, from about 7 to about 36, from about 7 to about 32, from about 7 to about 28, from about 7 to about 24, from about 7 to about 20 from about 7 to about 12, from about 7 to about 11.5, from about 7 to about 11, from about 7 to about 10.5, from about 7.5 to about 10.5, from about 8 to about 10.5, or from about 8 to about 10.

8. The catalyst composition of any one of claims 1 to 7, wherein a is from about 0.05 to about 3.5, from about 0.05 to about 3, from about 0.05 to about 2.5, from about 0.05 to about 2, from about 0.05 to about 1.5, from about 0.05 to about 1, from about 0.1 to about 1, from about 0.15 to about 1, from about 0.2 to about 1, from about 0.25 to about 1, from about 0.3 to about 1, from about 0.35 to about 1, from about 0.4 to about 1, from about 0.05 to about 1.25, from about 0.05 to about 0.75, from about 0.05 to about 0.5, from about 0.05 to about 0.4, from about 0.05 to about 0.3, from about 0.05 to about 0.2, or from about 0.05 to about 0.1.

9. The catalyst composition of any one of claims 1 to 4, wherein b is from about 0.3 to about 3, from about 0.4 to about 3, from about 0.5 to about 3, from about 0.75 to about 3, from about 1 to about 3, from about 1.5 to about 3, from about 2 to about 3, from about 2.5 to about 3, from about 0.75 to about 2.5, or from about 1 to about 2.5.

10. The catalyst composition of anyone of claims 5 to 8, wherein b is from about 0.01 to about 2.5, from about 0.01 to about 2, from about 0.01 to about 1.5, from about 0.02 to about 1.5, from about 0.04 to about 1.5, from about 0.06 to about 1.5, from about 0.08 to about 1.5, from about 0.1 to about 1.5, from about 0.2 to about 1.5, from about 0.3 to about 1.5, from about 0.4 to about 1.5, from about 0.5 to about 1.5, from about 0.1 to about 3, from about 0.2 to about 3, from about 0.3 to about 3, from about 0.4 to about 3, from about 0.5 to about 3, from about 0.75 to about 3, from about 1 to about 3, from about 1.5 to about 3, from about 2 to about 3, from about 2.5 to about 3, from about 0.75 to about 2.5, or from about 1 to about 2.5.

11. The catalyst composition of any one of claims 1 to 10, wherein c is from about 0.05 to about 4, from about 0.1 to about 4, from about 0.15 to about 4, from about 0.5 to about 4, from about 0.25 to about 4, from about 0.3 to about 4, from about 0.35 to about 4, from about 0.4 to about 4, from about 0.45 to about 4, from about 0.5 to about 4, from about 0.75 to about 4, from about 1 to about 4, from about 1 to about 3.5, from about 1 to about 3, from about 0.01 to about 3.5, from about 0.01 to about 3, from about 0.01 to about 2, from about 0.01 to about 1, from about 0.01 to about 0.5, from about 0.01 to about 0.4, from about 0.01 to about 0.3, from about 0.01 to about 0.2, from about 0.01 to about 0.1, or from about 0.01 to about 0.05.

12. The catalyst composition of any one of claims 1 to 11, wherein d is from about 0.01 to about 1.5, from about 0.01 to about 1, from about 0.02 to about 1, from about 0.04 to about 1, from about 0.06 to about 1, from about 0.08 to about 1, from about 0.1 to about 1, from about 0.15 to about 1, from about 0.15 to about 0.5, from about 0.02 to about 2, from about 0.05 to about 2, from about 0.1 to about 2, from about 0.5 to about 2, from about 1 to about 2, or from about 1.5 to about 2.

13. The catalyst composition of any one of claims 1 to 12, wherein e is from about 0.01 to about 1.5, from about 0.01 to about 1, from about 0.01 to about 0.5, from about 0.01 to about 0.4, from about 0.01 to about 0.3, from about 0.01 to about 0.3, from about 0.02 to about 0.3, from about 0.04 to about 0.3, from about 0.06 to about 0.3, from about 0.08 to about 0.3, from about 0.1 to about 0.5, or from about 0.2 to about 0.4.

14. The catalyst composition of any one of claims 1 to 13, wherein f is from about 0.02 to about 10, from about 0.04 to about 10, from about 0.06 to about 10, from about 0.08 to about 10, from about 0.1 to about 10, from about 0.2 to about 10, from about 0.4 to about 10, from about 0.6 to about 10, from about 0.8 to about 10, from about 1 to about 10, from about 1.5 to about 10, from about 2 to about 10, from about 2.5 to about 10, from about 3 to about 10, from about 3.5 to about 10, from about 4 to about 10, from about 4.5 to about 10, from about 5 to about 10, from about 0.01 to about 8, from about 0.01 to about 7, from about 0.01 to about 6, from about 0.01 to about 5, from about 0.01 to about 4, from about 0.01 to about 3, from about 0.01 to about 2, from about 0.01 to about 1, from about 0.01 to about 0.5, from about 0.01 to about 0.25, from about 0.01 to about 0.2, from about 0.01 to about 0.1, or from about 0.01 to about 0.05.

15. The catalyst composition of any one of claims 1 to 14, wherein g is from about 0.01 to about 0.2, from about 0.02 to about 0.2, from about 0.04 to about 0.2, from about 0.06 to about 0.2, from about 0.08 to about 0.2, or from about 0.1 to about 0.2.

16. The catalyst composition of any one of claims 1 to 14, wherein g is from about 0 to about 0.15, from about 0 to about 0.1, from about 0 to about 0.05, from about 0 to about 0.04, from about 0 to about 0.03, from about 0 to about 0.02, or from about 0 to about 0.01.

17. The catalyst composition of any one of claims 1 to 16, wherein A is selected from the group consisting of potassium, rubidium, and cesium.

18. The catalyst composition of any one of claims 1 to 16, wherein A is selected from the group consisting of rubidium and cesium.

19. The catalyst composition of any one of claims 1 to 18 further comprising a support.

20. The catalyst composition of claim 19, wherein the support comprises from about 30 wt. % to about 70 wt. %, from about 35 wt. % to about 70 wt. %, from about 40 wt. % to about 70 wt. %, from about 40 wt. % to about 65 wt. %, from about 40 wt. % to about 60 wt. %, from about 40 wt. % to about 55 wt. %, or from about 45 wt. % to about 55 wt. % of the catalyst composition.

21. A process for the ammoxidation of an olefin, the process comprising:
reacting in the vapor phase at an elevated temperature and pressure the olefin with a molecular oxygen containing gas and ammonia in the presence of the catalyst composition of any one of claims 1 to 20.

22. The process of claim 21, wherein the olefin is selected from the group consisting of propylene, isobutylene or mixtures thereof to produce a reaction product comprising acrylonitrile, methacrylonitrile and mixtures thereof, respectively.

23. The process of claim 21 or claim 22, wherein the molar ratio of ammonia to olefin is from about 0.5:1 to about 2:1, from about 0.5:1 to about 1.5:1, from about 0.5:1 to about 1.4:1, from about 0.5:1 to about 1.3:1, from about 0.5:1 to about 1.2:1, from about 0.5:1 to about 1.1:1, from about 0.5:1 to about 1:1, or from about 0.75:1 to about 1:1.

24. The process of any one of claims 21 to 23, wherein the olefin comprises propylene and the molar ratio of ammonia to propylene is from about 0.9:1 to about 1.3:1, from about 0.9:1 to about 1.2:1, from about 0.9:1 to about 1.1:1, from about 1:1 to about 1.1:1, or from about 1:1 to about 1.05:1.

25. The process of any one of claims 21 to 24, wherein the olefin comprises propylene and the source of oxygen comprises air, and wherein the molar ratio of air to propylene is from about 5:1 to about 20:1, from about 5:1 to about 15:1, from about 6:1 to about 12:1, from about 7:1 to about 15:1, from about 8:1 to about 15:1, from about 8:1 to about 14:1, from about 8:1 to about 13:1, or from about 8:1 to about 12:1.

26. The process of any one of claims 21 to 25, wherein the olefin comprises propylene and the olefin conversion is about 85% or greater, about 86% or greater, about 87% or greater, about 88% or greater, about 89% or greater, about 90% or greater, about 91% or greater, about 92% or greater, about 93% or greater, about 94% or greater, about 95% or greater, about 96% or greater, about 97% or greater, about 98% or greater, about 99% or greater, about 99.1% or greater, about 99.2% or greater, about 99.3% or greater, about 99.7% or greater, about 99.8% or greater, about 99.6% or greater, about 99.7% or greater, about 99.8% or greater, or about 99.9% or greater.

27. The process of any one of claims 21 to 26, wherein the olefin comprises propylene and the nitrile yield is about 70% or greater, about 72% or greater, about 74% or greater, about 76% or greater, about 78% or greater, about 80% or greater, about 82% or greater, about 84% or greater, about 86% or greater, about 88% or greater, or about 90% or greater.
